(19) United States
(12) Patent Application Publication
(10) Pub. No.: US 2024/0390117 A1
(43) Pub. Date: Nov. 28, 2024

(54) DENTAL PROSTHETIC APPLIANCE

(71) Applicant: Opa Dental, LLC, Huntington, WV (US)

(72) Inventors: Lee Gorham, Huntington, WV (US); Strat Fotos, Huntington, WV (US)

(73) Assignee: Opa Dental, LLC, Huntington, WV (US)

(21) Appl. No.: 18/613,368

(22) Filed: Mar. 22, 2024

Related U.S. Application Data
(60) Provisional application No. 63/468,430, filed on May 23, 2023.

(51) Int. Cl. A61C 8/00; A61C 13/01; A61K 6/818; A61K 6/887

(52) U.S. Cl. CPC A61C 8/0093; A61C 8/0012; A61C 8/0069; A61C 13/01; A61K 6/818; A61K 6/887

(57) ABSTRACT

The present invention relates to a bridge style dental prosthetic for replacing multiple lost or damaged teeth. More specifically, the present invention relates to an appliance comprising a base made of a first non-compressive material that is indefinitely affixed to the patient's jaw and a dentition portion that is made of a second compressive material and is removably affixed to the base portion, thus allowing restoration and/or replacement of the dentition portion without the need to remove and/or replace the base portion. The invention may include a dentition channel adapted to receive and removably secure the dentition to the base. The invention may also include a post disposed in the lower surface of the dentition that is received by an opening in the distal surface of the base to removably secure the dentition to the base.

### BACKGROUND OF THE INVENTION

### 1) Field of the Invention

The present invention relates to a bridge style dental prosthetic for replacing multiple lost or damaged teeth. More specifically, the present invention relates to an appliance comprising a base made of a first non-compressive material that is affixed to an implant in the patient's jaw and a dentition portion that is made of a second compressive material and is removably affixed to the base portion, thus allowing restoration and/or replacement of the dentition portion without the need to replace the base portion and in some case without the need to remove the base portion from the patient's jaw.

### 2) Description of Related Art

Prior art appliances, such as the one shown in FIG. 1A consist of a one-piece appliance 100 formed from single material. This poses at least two different problems that the present invention addresses. First, it is well known that it is preferable to have dentition 102 that is formed from a compressive material that will absorb some of the shock from biting and/or chewing by the patient. The use of a softer material helps absorb the shock from the patient's biting and/or chewing, which would otherwise be transferred to the implant (not shown) that secures the appliance 100 to the patient's jaw. Over time, this shock could damage and/or compromise the implant and/or its positioning in the patient's jaw. Over time, this shock could also cause the periodontal ligament to cease functioning properly. The use of a softer dentition material provides an appropriate amount of shock absorption to prevent these problems from arising. Moreover, this softer dentition material could also accommodate patients experiencing temporomandibular disorder by alleviating some of the pressures and shock that would be placed on the temporomandibular joints by prior art dentition materials.

Another advantage of the softer material is that it slows and/or prevents excessive wear on the opposing dentition that would occur when a harder material is used to form the dentition. However, the problem with using a softer material for the dentition is that it is more likely to become damaged or worn, thus requiring replacement. In fact, this softer material is designed to wear and be replaced. Because one-piece appliances 100 are designed to be permanently and/or indefinitely affixed to the patient's jaw, replacement of the dentition proves to be very expensive and requires a lengthier, more involved, and more expensive procedure that can be painful to the patient. Therefore, one-piece appliances often use a harder material for both the dentition 102 and the base portion 104 of the one-piece appliance.

Professionals in the dental industry have long been searching for a substitute for dentition made of hard material such as zirconia that might address the premature or unnatural wear of natural dentition (i.e. the patient's teeth) that oppose the appliance and/or dentition used therein as well as the additional and/or unnecessary shock and/or impact that is placed on the implant securing the appliance to the patient's jaw. Professionals have similarly long been looking for a substitute that would prevent premature displacement, wear and/or failure of the implants to which the appliance is secured.

Other one-piece appliances in the prior art, such as the one shown as 110 in FIG. 1B, use a softer material for dentition 112 that is permanently glued to the base 114. Because this appliance is permanently or indefinitely affixed to the patient's jaw, a full appliance replacement is required to replace the dentition. Again, professionals have long been searching for an appliance that allows replacement of the dentition, without the need to replace the base carrying the dentition.

Other appliances include dentures that are not affixed to the patient's jaw and instead rest on the patient's jaw so that the entire appliance may be easily replaced. The difficulty with a denture appliance is that it requires daily upkeep to ensure that it stays clean of bacteria and the like, which can form between the appliance and the patient's jaw.

Accordingly, it is an object of the present invention to provide a prosthetic appliance that includes a base made of a first hard and resilient material and is affixed to the patient's jaw and a dentition that is made of a second compressive material that is removably affixed to the base.

### SUMMARY OF THE INVENTION

The above objectives are accomplished according to the present invention by providing a dental prosthetic appliance comprising: a base comprising a first material, the base having a proximal surface that is adapted to be secured to an implant disposed in a patient's jaw and a distal surface that is adapted to receive a dentition; a dentition comprising a second material, the dentition having a lower surface that is adapted to be removably secured to the distal surface said base, an upper surface, an inner surface and an outer surface; and, a dentition channel that disposed in at least a portion of the distal surface of the base and is adapted to receive the dentition, wherein the dentition channel includes a bottom surface, a first side and a second side so that when the dentition channel receives the dentition, the bottom surface of the dentition channel is adjacent to the lower surface of the dentition, the first side extends along a portion of the outer surface of the dentition towards the upper surface of the dentition and the second side extends along a portion of the inner surface of the dentition towards the upper surface of the dentition.

In one embodiment, the first material of the base has a flexural strength of at least 1000 megapascals and may contain at least 80% by weight zirconia dioxide. The second material of the dentition may have a flexural strength of between 70-250 megapascals and may contain at least 90% by weight of polymethyl methacrylate.

The present invention may further comprise an implant housing for receiving an implant to secure the base to the patient's jaw, wherein the implant housing extends upwardly from the bottom surface of the dentition channel so that an outer surface of the implant housing may be laterally spaced from the first side and the second side of the dentition channel by 2.5 mm or more. The implant housing may also include an inner that forms a conduit through which the implant may pass. The implant housing may have a top portion that is vertically spaced from the bottom surface of the dentition channel and a base surface that is adjacent to the bottom surface of the dentition channel wherein the inner surface at the top portion may have a diameter that is less than the diameter of the inner surface at the base portion.

The present invention may further comprise a protuberance a protuberance disposed on the lower surface of the dentition wherein the protuberance carries that is adapted to be received by the dentition channel. In one embodiment the present invention comprises a first protuberance and a second protuberance, each of which is disposed on the lower surface of the dentition and are adapted to be received by the dentition channel, wherein the first protuberance is laterally spaced from the second protuberance by a distance that is at least as great as a width of the implant housing.

In one embodiment, the second side of the dentition channel extends upwardly from the bottom surface of the dentition channel by a height that is at least 25% of the height of the dentition when measured from the lower surface to the upper surface of the dentition.

The present invention may further comprise a first dentition indent defined by a portion of the first side and a portion of the second side of the dentition channel wherein the portion of the first side has a first radiused surface and the portion of the second side has a second radiused surface that opposes the first radiused surface so that when the dentition is received by the dentition channel, the first radiused surface is may engage the outer surface of the dentition and the second radiused surface may engage the inner surface of the dentition. The present invention may further comprise a second dentition indent that is adjacent to the first dentition indent so that the first side of the dentition channel includes a third radiused surface that is adjacent to the first radiused surface, so that when the dentition is received by the dentition channel, the first radiused surface engages the buccal surface of the first tooth and the third radiused surface engages the buccal surface of a second tooth.

The present invention may further comprise a plurality of securing posts disposed on the lower surface of the dentition and a plurality of corresponding post receiving openings defined in the distal surface of the base and that are adapted to receive the plurality of corresponding securing posts and, thus, removably secure the dentition to the base when the dentition is received by the dentition channel.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIGS. 1A-1B show prior art appliances.

FIG. 2A shows a perspective view of an embodiment of the present invention.

FIGS. 2B-2C show exploded views of an embodiment of the present invention.

FIG. 2D shows a top plan view of the dentition and base that is included an embodiment of the present invention.

FIG. 2 E shows perspective view of a tooth included in an embodiment of the invention.

FIG. 3 A shows a top plan view of the dentition and base that is included in an embodiment of the present invention.

FIG. 3B shows a perspective view of an implant housing that is included in an embodiment of the present invention.

FIG. 3C shows a perspective view of the base that is included in an embodiment of the present invention.

FIGS. 3D-E show a perspective view of an embodiment of the invention.

FIG. 4 shows an exploded view of securing members used in an embodiment of the present invention.

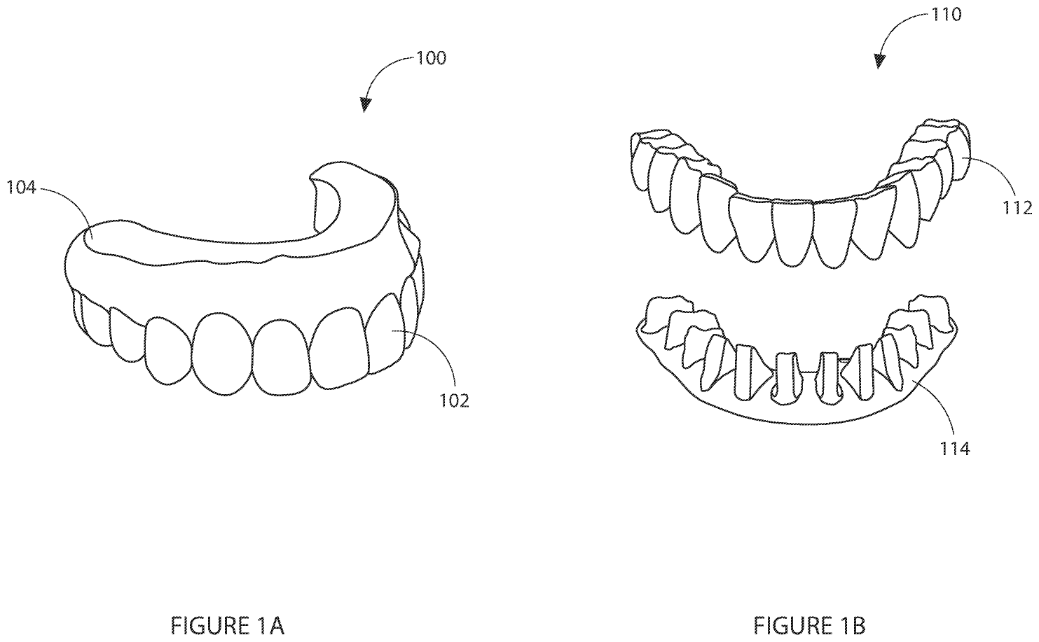

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or in testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For purposes of describing the invention, proximal surface shall refer to an intaglio surface that, when compared to the “distal surface,” is closer to (or at times, in contact with) the patient's jaw to which the appliance will be attached. The distal surface refers to an occlusal surface that when compared to the proximal surface is further away from the patient's jaw to which the appliance will be attached. In some cases, the proximal surface (such as the lower or proximal surface of the base) shall be the surface that contacts the patient's jaw when installed. In other cases, the proximal surface (such as for the dentition), may not actually contact the patient's jaw but the proximal surface will be closer to the jaw on which the appliance is installed than the distal/upper surface of the dentition that is used for chewing. Similarly, an outer or exterior surface shall refer to a surface that is closest to the patient's lips and/or cheek when the appliance is installed, such as a buccal or facial surface. An inner or interior surface shall refer to a lingual surface that is closest to the patient's tongue when the appliance is installed. In the shown embodiments, the outer surface generally corresponds with the convex surface, whereas the inner surface generally corresponds with the concave surface. Where the appliance is not a full bridge, however, it may not have a convex and/or concave surface that correspond to the inner and outer surface respectively.

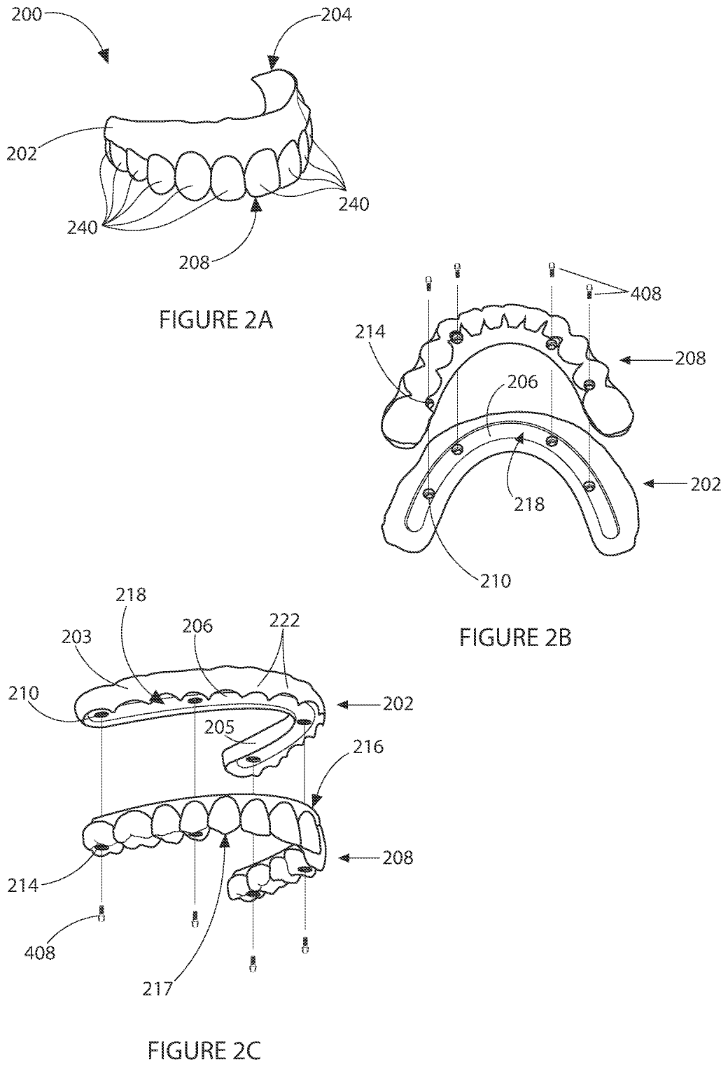

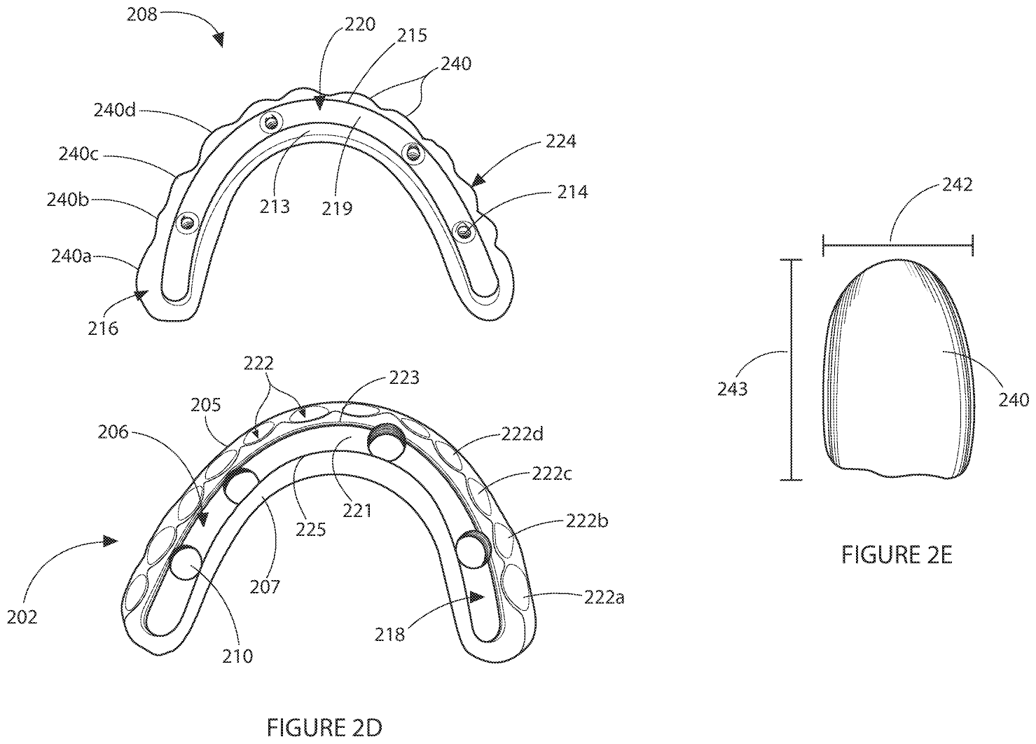

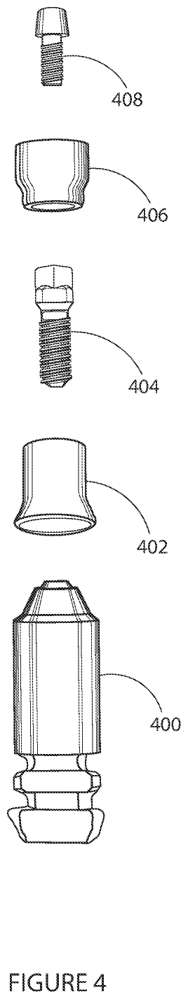

Referring now to FIGS. 2A-D, an embodiment of the present invention will be further disclosed. Appliance 200 includes a base 202 having a lower and/or proximal surface 204 that is adapted to fit onto a patient's jaw and a distal surface 206 that is adapted to receive dentition 208. The proximal surface 204 includes securing openings 210 that are adapted to allow the base 202 to be secured to an implant 400 (see FIG. 4) that is implanted into the patient's jaw. In one embodiment, the securing openings 210 extend from the base's intaglio/proximal surface 204 through the base's occlusal/distal surface 206. Once the implant 400 is placed in the patient's jaw, a coupling 402 can be used such that the coupling would extend from the implant through the securing openings defined in the base. A base securing member 404 may be received by the implant and used to secure the base to the patient's jaw as is typically done in the prior art.

In the embodiment shown in FIGS. 2A-C, the distal surface 204 of the base 202 includes a dentition securing channel 218 that is adapted to receive a protuberance 220 disposed on the dentition's proximal surface 216. The securing channel 218 and corresponding protuberance 220 could extend along the entire length of the appliance or in one or more areas or portions. In at least one embodiment, the securing channel includes a bottom surface 221, a first side 223 and a second side 225. In the shown embodiment, the first and second sides both extend upwardly from the bottom surface at a 90 degree angle to create a square shaped well into which the protuberance 220 may be received. In alternate embodiments, the first and second side could extend upwards from the base at different angles but preferably the first and second sides would extend parallel to one another. In at least one embodiment, the second wall 225 extends from the bottom surface 221 by a distance that is at least 25% of the height 243 of the corresponding tooth 240. The protuberance 220 includes a bottom surface 219, a first side 215 and a second side 213, which form a shape that is adapted to be received by the dentition securing channel and engaged by the channels bottom and side surfaces. In at least one embodiment, the protuberance and the dentition securing channel are adapted so that when the protuberance is received by the securing channel the protuberance's bottom surface 219 does not contact the bottom surface 321 of the dentition securing channel 318. As is explained later, this vertical spacing which may be a little as 0.1 mm, allows room for an adhesive and/or cement to bond the protuberance within the dentition securing channel.

The base 202 may also include dentition indents 222 that are disposed adjacent to the securing channel 218. In one embodiment, the dentition idents are vertically spaced from the bottom of the securing channel. Dentition indents 222 are adapted to receive and engage the dentition's outer surface 224. When dentition 208 is placed in and/or secured to the base 202 so that the protuberance 220 is received by the dentition channel 218, each of the plurality of teeth 240 defined by the dentition 208 will be engaged by the corresponding dentition indent. The indents 222 help secure the dentition in place and prevent shifting, improve the appearance of the appliance to make it appear more like natural teeth and gums and help prevent materials and/or bacteria from gaining access between the dentition's proximal surface 216 and the base's distal surface 204.

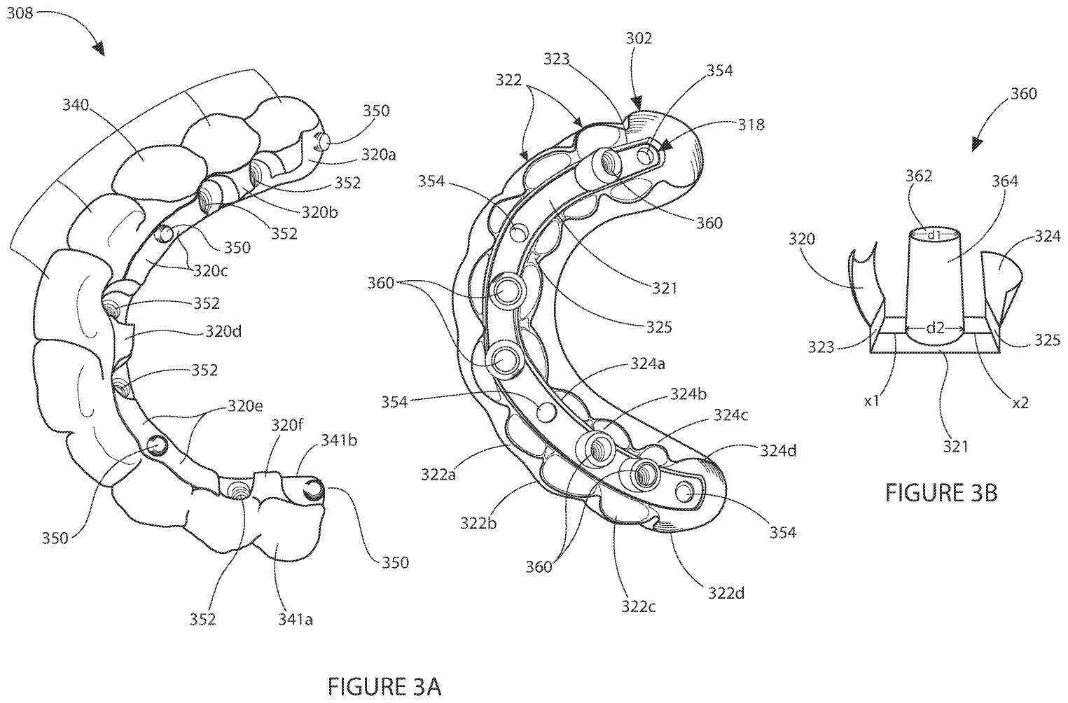

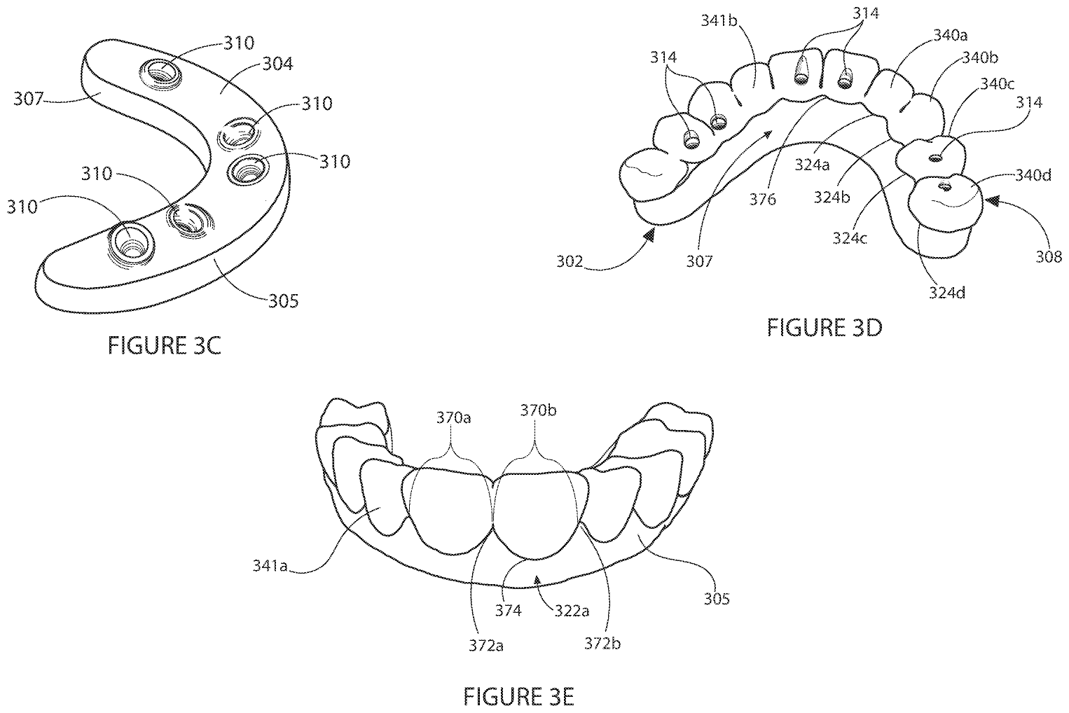

In one embodiment, the dentition indent comprises a first horizontally radiused surface 222a-d that is adjacent to and/or vertically spaced from the first wall of the dentition securing channel. The dentition indent could also include a second horizontally radiused surface (shown in FIG. 3A as 324a-d) that is adjacent to and/or vertically spaced from the second side of the dentition channel 218 so that the second radiused surface 324a-d opposes the corresponding first radiused surface 222a-d. In alternate embodiments, the first and second radiused surfaces could be formed in the first side 223 and second side 225 of the dentition channel, respectively, as opposed to being disposed adjacent to them. As shown in FIG. 2D, each of the horizontally radiused surfaces is adapted to correspond to and engage the curvature of a tooth 240 that is to be received by the respective dentition indent 222 when the horizontally radiused surface extends along the tooth's width 242. In yet another embodiment, the first radiused surface 222a-d and second radiused surface 324a-d could also be vertically radiused so that they correspond with and are adapted to engage the curvature of the respective tooth to be received by that dentition indented when the vertically radiused surface extends along the tooth's length 243. In any of the above embodiments, the first radiused surface 22a-d of the dentition indent that is adjacent to or formed within the first side of the dentition securing channel is adapted to engage the outer/buccal surface of the tooth 240 to be received by the respective dentition indent 222. The second radiused surface of the dentition indent 342a-d is adapted to engage the inner/lingual surface of the tooth 240 to be received by the respective dentition indent 222.

Referring now to FIGS. 3A-E, another embodiment of the present invention is shown. Base 302 includes the dentition channel 318, which includes a bottom surface 321, a first side 323 and a second side 325. The base further includes a plurality of dentition indents 322 that are adapted to receive and engage a corresponding tooth 340 that is defined by the dentition 308. The dentition indent may include a first radiused surface 322a that is disposed adjacent to or formed in the first side 323 of the dentition channel. This first radiused surface is adapted to engage the outer/buccal surface 341a of the tooth. The second radiused surface 324a is disposed adjacent to or formed in the second side 325 of the dentition securing channel 318. This second radiused surface is adapted to engage the inner/lingual surface 341b of a corresponding tooth that is to be received by the dentition indent. The curvature of the first radiused surface 322a may be the same as or different than the curvature of the second surface 324a. In at least one embodiment, the base includes a plurality of dentition idents 322. In such an embodiment a second dentition indent may have a third radiused surface 322b that is adjacent to or formed in the first wall 322 of the dentition securing channel at a location that may be adjacent to the first radiused surface 322a. Similarly, the second dentition indent may have a fourth radiused surface 324b that opposes the third radiused surface 322b. This fourth radiused surface may be adjacent to or formed in the second wall 325 of the dentition securing channel and may be adjacent to the first dentition indent so that the fourth radiused surface is adjacent to the second radiused surface.

As shown in FIGS. 3A-C, the base further includes a plurality of implant housings 360 disposed in the dentition securing channel 318. The implant housing forms a conduit that allows the base securing member 404 to pass through the implant housing and through the securing openings 314 formed in the base so that the securing member may secure the base to an implant in a patient's jaw. The implant housing 30 may have any number of cross-sectional shapes. In at least one embodiment, the implant housing has a conical or frustro-conical shape, where the inner surface 362 at a top portion of the housing has a smaller diameter d1 than the diameter d2 of the inner surface at a bottom portion of the implant housing. In one embodiment, d1 is 4 millimeters and d2 is 5 millimeters. In the shown embodiment, the implant house is disposed in the dentition securing channel so that the housing's outer surface 364 is laterally spaced from the dentition channel's first side 323 by a distance of x1 and is laterally spaced from the dentition channel's second side 325 by a distance of x2. In one embodiment, distance x1 and x2 are equal to 2.5 millimeters. In alternate embodiments, x1 could be a distance that is different than x2 and both x1 and x2 could be in the range of 1.5-6 millimeters.

The dentition 308 defines a plurality of teeth 340a-d, each having an outer/buccal surface 341a and an inner/lingual surface 341b. Each of the teeth correspond to a dentition indent 322 and the curvature of radiused surfaces that are included in the corresponding dentition indent match the curvature of the radiused surface of the tooth. The dentition may include a plurality of protuberances 320a-g, each of which are adapted to be received by the dentition channel 318. The protuberances are laterally spaced by an implant housing receptacle 352 that is adapted to receive the implant housing 360 when the dentition is inserted into the dentition securing channel 318. One ore more of the protuberances may include a securing post 350 disposed thereon that is adapted to be received by a corresponding securing post receiving opening 354 that is disposed in the bottom surface 321 of the dentition securing channel 318. In one embodiment, the posts have a height of 2.0 millimeters and a diameter of 2.5 millimeters. In one embodiment, the securing post extends from the protuberance's bottom surface 219 at a 5 degree tilt so that the tip of the securing post 350 tilts toward the lingual/inner surface of the dentition/tooth.

Referring now to FIGS. 3D-E, the present invention can be seen where the dentition 308 is removably secured to the base 302. A plurality of teeth 340a-d are received and engaged by a plurality of dentition indents 322a-d so that the second radiused surfaces 324a-d of each respective indent engages an inner/lingual surface 341b of each corresponding tooth. The base further includes an interior/lingual surface 307 (also shown as 207 in FIG. 2D) and exterior/buccal surface 305 (also shown as 205 in FIG. 2D). The thickness of both the interior surface 307 and exterior surface 305 taper in thickness when measured from the proximal/intaglio surface 304 (also shown as 204 in FIG. 2A) to the distal surface 306 (also shown as 206 in FIGS. 2A and 2D). This tapering helps to create a smooth finish line between the tooth and the interior surface 307/exterior surface 305 that minimizes any gaps between the dentition and the base where debris or bacteria could become lodged.

As can be seen in FIG. 3E, the dentition indent 322 can further include a plurality of dentition arches 370a and 370b. Each of the dentition arches includes a first papilla 372a and a second papilla 372b interconnected by the dentition indent's first radiused surface 322a. The first radiused surface has a parabolic curvature where the apex of the parabola 374 is located at the midpoint of the tooth's width (shown as 242 in FIG. 2E). In one embodiment, the distance between the tip of the papilla 372a-b and the bottom surface 321 of the dentition channel 318 is at least 50% of the distance between the bottom surface 321 and the top of the opposing second radiused surface 376 of the same dentition indent, which is also known as the finish line. In one embodiment, the distance between the bottom surface and the apex 374 is at least 25% of the distance between the bottom surface 321 and the top of the opposing second radiused surface 376 of the same dentition indent.

Prior to placing the dentition 308 into the base 308, an adhesive or cement is placed on the proximal surface of the dentition, including on the protuberances 320a-g and securing posts 350. Once the dentition 308 is placed into the base 302 so that the protuberances 320a-g are received by the dentition securing channel 318, the securing posts 350 are received by the post securing openings 354 and the teeth 340a-d are received by the dentition indents 322a-d, and additional cement, adhesive or sealant may be used to seal any gap between the dentition and the external surface 305 and/or internal surface 307 of the base at the finish line where they meet. This cement and/or sealant is generally known in the art and helps prevent debris or bacteria from becoming lodged between the dentition and the base.

Referring to FIGS. 2A-C, 3D, and 4, the dentition 208/308 may be removably secured to base 202/302 by way of a dentition securing member 408 that is adapted to be received and securely engaged by the base securing member 404. The dentition may include dentition securing conduits 214 extending from an upper opening in the dentition's distal surface 217 to a lower opening in the dentition's proximal surface 216. The dentition securing member 408 may be inserted into the dentition securing conduit so that it may be securely engaged by the base securing member 404. In one embodiment a securing collar 406 may be disposed between base securing member 404 and the dentition securing member 408. The collar 406 may help secure the base to the patient's jaw when the base securing member 404 is securely fastened to the implant 300.

In one embodiment, once the dentition 208 is secured to the base, a closure cap may be placed in the upper opening of the securing conduit 214/314 defined in the dentition's inner/lingual surface 217. This cap helps keep materials and bacteria from gaining access to the securing conduit 214. The cap may be pre-formed and inserted into the opening or it may be a cement or the like that is poured into the opening.

In any of the embodiments discussed herein, once base 202 is secured to the patient's jaw, the dentition 208 may be removably secured to the base's distal surface 206. In another embodiment, the detention may be removably secured to the base's distal surface prior to installation of the base onto the patient's jaw.

Because the base portion 202 is meant to be affixed to the patient's jaw indefinitely, it may be made of a first durable, non-compressive material such as Zirconium dioxide (ZrO₂) or any other similar material generally known in the art. In at least one embodiment, the base is formed of a material marketed under the name LUNA dental zirconia. However, any material having a flexural strength of at least 800 megapascals will suffice. Typically, a material having at least 80% by weight of zirconia dioxide will have sufficient flexural strength to serve as the first material for the base.

To cause the dentition to absorb shock in the same manner as the patient's periodontal ligaments, the dentition is made of a second material that is more compressive than the first material. It is important that the second material be soft enough to absorb a sufficient level of shock while chewing but be hard enough to prevent premature wear or failure of the dentition. This second material could include materials such as polymethyl methacrylate (PMMA) or any other similar material generally known in the art. However, any material that provides a flexural strength between 70-120 megapascals would be sufficient to serve as the second material for the dentition. Typically, materials that include at least 90% of PMMA will provide a sufficient flexural strength range. As the dentition wears, becomes damaged or requires repair or replacement, the closure cap may be removed (by drilling or otherwise) from the dentition securing opening 314 to expose the dentition securing member 308. The dentition securing members may then be detached and/or removed from the base securing members for repair or replacement, thereby allowing the base member to remain indefinitely affixed to the patient's jaw.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A dental prosthetic appliance comprising:
A base comprising a first material, the base having a proximal surface that is adapted to be secured to an implant disposed in a patient's jaw and a distal surface that is adapted to receive a dentition;
A dentition comprising a second material, the dentition having a lower surface that is adapted to be removably secured to the distal surface said base, an upper surface, an inner surface and an outer surface; and,
A dentition channel that disposed in at least a portion of the distal surface of the base and is adapted to receive the dentition, wherein the dentition channel includes a bottom surface, a first side and a second side so that when the dentition channel receives the dentition, the bottom surface of the dentition channel is adjacent to the lower surface of the dentition, the first side extends along a portion of the outer surface of the dentition towards the upper surface of the dentition and the second side extends along a portion of the inner surface of the dentition towards the upper surface of the dentition.

2. The appliance of claim 1 wherein the first material of the base has a flexural strength of at least 800 megapascals.

3. The appliance of claim 1 wherein the first material of the base contains at least 90% by weight zirconia dioxide.

4. The appliance of claim 1 wherein the second material of the dentition has a flexural strength of at least 70 megapascals but no greater than 250 megapascals.

5. The appliance of claim 1 wherein the second material of the dentition least 90% by weight polymethyl methacrylate.

6. The appliance of claim 1 further comprising an implant housing for receiving an implant abutment, wherein the implant housing extends upwardly from the bottom surface of the dentition channel so that an outer surface of the implant housing is laterally spaced from the first side and the second side of the dentition channel and an inner surface of the implant housing forms a conduit through which the implant may pass.

7. The appliance of claim 6 wherein the implant housing has a top portion that is vertically spaced from the bottom surface of the dentition channel and a base surface that is adjacent to the bottom surface of the dentition channel wherein the inner surface at the top portion has a diameter that is less than the diameter of the inner surface at the base portion.

8. The appliance of claim 1 wherein the second side of the dentition channel extends upwardly from the bottom surface of the dentition channel by a height that is at least 25% of the height of the dentition when measured from the lower surface to the upper surface of the dentition.

9. The appliance of claim 1 further comprising a first dentition indent defined by a portion of the first side and a portion of the second side of the dentition channel wherein the portion of the first side has a first radiused surface and the portion of the second side has a second radiused surface that opposes the first radiused surface so that when the dentition is received by the dentition channel, the first radiused surface is adapted to engage the outer surface of the dentition and the second radiused surface is adapted to engage the inner surface of the dentition.

10. The appliance of claim 1 further comprising a post disposed on the lower surface of the dentition and a post receiving opening defined in the distal surface of the base and adapted to removably secure the dentition to the base.

11. A dental prosthetic appliance comprising:
A base comprising a first material, the base having a proximal surface that is adapted to be secured to an implant disposed in a patient's jaw and a distal surface that is adapted to receive a dentition;
A dentition defining a plurality of teeth comprising a second material, the dentition having a lower surface that is adapted to be removably secured to said distal surface of the base, an upper surface defining a chewing surface for the plurality of teeth, an inner surface forming a lingual surface for the plurality of teeth and an outer surface defining a buccal surface for the plurality of teeth;
A dentition channel that is disposed in at least a portion of the distal surface of the base and is adapted to receive the dentition, wherein the dentition channel includes a bottom surface, a first side and a second side so that when the dentition channel receives the dentition, the bottom surface of the dentition channel is adjacent to the lower surface of the dentition, the first side extends along a portion of the outer surface of the dentition towards the upper surface of the dentition and the second side extends along a portion of the inner surface of the dentition towards the upper surface of the dentition; and,
a first dentition indent defined by a portion of the first side and a portion of the second side of the dentition channel wherein the portion of the first side has a first horizontally radiused surface corresponding to the radius of the buccal surface of a first tooth and the portion of the second side has a second horizontally radiused surface that opposes the first radiused surface and corresponds to the radius of the lingual surface of the first tooth so that when the dentition is received by the dentition channel, the first radiused surface is adapted to engage the buccal surface of the first tooth and the second radiused surface is adapted to engage the lingual surface of the first tooth.

12. The appliance of claim 11 wherein the first material of the base has a flexural strength of at least 1000 megapascals.

13. The appliance of claim 11 wherein the second material of the dentition has a flexural strength that is within the range of no less than 100 megapascals and no greater than 240 megapascals.

14. The appliance of claim 11 further comprising a second dentition indent that is adjacent to the first dentition indent so that the first side of the dentition channel includes a second radiused surface that is adjacent to the first radiused surface, so that when the dentition is received by the dentition channel, the first radiused surface engages the buccal surface of the first tooth and the second radiused surface engages the buccal surface of a second tooth.

15. The appliance of claim 11 further comprising:
an implant housing for receiving an implant abutment, wherein the implant housing extends upwardly from the bottom surface of the dentition channel; and
a first protuberance and a second protuberance, each of which is disposed on the lower surface of the dentition and are adapted to be received by the dentition channel, wherein the first protuberance is laterally spaced from the second protuberance by a distance that is at least as great as a width of the implant housing.

16. A dental prosthetic appliance comprising:
A base comprising a first material, said base having a proximal surface that is adapted to be secured to an implant disposed in a patient's jaw and a distal surface that is adapted to receive a dentition;
A dentition comprising a second material, said dentition having a lower surface that is adapted to be removably secured to said distal surface said base, an upper surface, an inner surface and an outer surface;
A dentition channel that disposed in at least a portion of the distal surface of the base and is adapted to receive the dentition, wherein the dentition channel includes a bottom surface, a first side and a second side so that when the dentition channel receives the dentition, the bottom surface of the dentition channel contacts the lower surface of the dentition, the first side extends along a portion of the outer surface of the dentition towards the upper surface of the dentition and the second side extends along a portion of the inner surface of the dentition towards the upper surface of the dentition; and,
A plurality securing posts of disposed on the lower surface of the dentition and a plurality of corresponding post receiving openings defined in the distal surface of the base and adapted receive the plurality of securing posts to removably secure the dentition to the base.

17. The appliance of claim 16 further comprising a dentition arch formed in a portion of the first side of the dentition channel, the arch including two papilla that extend from the bottom surface of the dentition channel by a distance that is at least 50% of the distance by which the second side of the dentition channel extends from the bottom surface.

18. The appliance of claim 16 further comprising a protuberance disposed on the lower surface of the dentition wherein the protuberance carries the plurality of securing posts and is adapted to be received by the dentition channel so that the securing posts are received by the securing post receiving openings.

19. The appliance of claim 18
an implant housing for receiving an implant abutment, wherein the implant housing extends upwardly from the bottom surface of the dentition channel; and
a second protuberance that is disposed on the lower surface of the dentition and is adapted to be received by the dentition channel, wherein the second protuberance is laterally spaced from the first protuberance by a distance that is at least as great as a width of the implant housing.

20. The appliance of claim 16 further comprising a first dentition indent defined by a portion of the first side and a portion of the second side of the dentition channel wherein the portion of the first side has a first horizontally radiused surface corresponding to the radius of the buccal surface of a first tooth defined by the dentition and the portion of the second side has a second horizontally radiused surface that opposes the first radiused surface and corresponds to the radius of the lingual surface of the first tooth so that when the dentition is received by the dentition channel, the first radiused surface is adapted to engage the buccal surface of the first tooth and the second radiused surface is adapted to engage the lingual surface of the first tooth.
